(19) United States
(12) Patent Application Publication
(10) Pub. No.: US 2001/0053384 A1
(43) Pub. Date: Dec. 20, 2001

(54) SITE-DIRECTED TRANSFECTION WITH ULTRASOUND AND CAVITATION NUCLEI

(76) Inventors: JAMES F. GREENLEAF, ROCHESTER, MN (US); MARK E. BOLANDER, ROCHESTER, MN (US); WILLIAM J. GREENLEAF, ROCHESTER, MN (US)

Correspondence Address: QUARLES & BRADY LLP, 411 E. WISCONSIN AVENUE, SUITE 2040, MILWAUKEE, WI, 53202-4497, US

(21) Appl. No.: 09/110,049

(22) Filed: Jul. 2, 1998

Related U.S. Application Data
(60) Provisional application No. 60/051,834, filed on Jul. 7, 1997.

(51) Int. Cl. A61N 1/18; A61H 5/00; A61N 1/38

(52) U.S. Cl. 424/450; 204/157.15; 600/455; 600/468; 600/434; 600/458; 601/2; 606/28; 607/24

(57) ABSTRACT

A method for the delivery of substances to a cell is disclosed. In a preferred embodiment, one administers continuous wave ultrasound or pulse-wave ultrasound to a cell bathed in a cocktail containing macromolecules and monitors the ultrasound using reflected echos of the ultrasound. One then observes incorporation of the substances into the cell. In a preferred embodiment of the present invention, macromolecules are combined in a cocktail solution comprising bubble micronuclei.

### CROSS-REFERENCE TO RELATED APPLICATION

This application claims the benefit of priority of U.S. Ser. No. 60/051,834.

### STATEMENT REGARDING FEDERALLY SPONSORED RESEARCH OR DEVELOPMENT

Not applicable.

### BACKGROUND OF THE INVENTION

Gene therapy is an exciting and burgeoning area of medicine. The basic concept of gene therapy is to introduce a good copy of a defective gene or, if possible, correct the defective gene. As it becomes clear that many diseases can be directly attributed to faulty genes, this means of therapy will gain even more importance. Already there are many diseases that are being targeted by this up-and-coming field. Severe combined immunodeficiency syndrome (SIDS), cystic fibrosis, and Gaucher's disease [1], are among the many diseases to which experimental gene therapy techniques are being applied in humans. A myriad of other diseases such as Tay-Sachs [2], and many forms of cancer [3, 1], seem fit for gene therapy techniques.

Gene therapy is based on deceiving the body's cells. Foreign DNA is placed into a target cell, and this cell expresses the DNA as if it were its own. With appropriate promoters and enhancers, the cellular machinery manufactures the protein that is coded on the foreign DNA. This foreign DNA specifically produces a protein that is expected to have therapeutic value. The uptake of foreign DNA by a cell is called transfection. Transfection occurs in two manners: transient and stable. Transient transfection occurs when the foreign DNA is expressed by the cell but is not incorporated into the nuclear DNA of the cell. Because of this lack of incorporation, the DNA is generally not passed to the daughter cells upon cell division. Stable transfection occurs when the foreign DNA is incorporated into the nuclear DNA of the cell and the genetic material is passed on to the daughter cells.

Gene therapy can take place in vivo or ex vivo. Generally, ex vivo methods involve harvesting a patient's affected cells, culturing them, transfecting the cells, and re-implanting the genetically altered cells in the patient's body [4]. In vivo transfection takes place entirely in the patient's body. The DNA is transferred to the affected cells while they are still within the patient [4]. This method is very attractive. Advantages that in vivo methods have over ex vivo include a less involved protocol and usually lower possibility of contamination. On the other hand, due to the fact that the patient's cells are much less accessible, many different techniques for transfer of genetic material are hindered. Acoustically induced transfection is among the procedures that are potentially well suited for in vivo transfection [5].

Many different techniques place foreign DNA into a target cell. These techniques can be divided into two broad categories; chemically mediated transfection and mechanically mediated transfection. Among the chemical techniques are calcium phosphate, viral encapsulation, and lipofection. The calcium phosphate method of transfection uses calcium phosphate to precipitate DNA onto the cells where the complexes are absorbed through the membrane. Viral particles engineered to carry foreign DNA are increasingly being used to deliver DNA into cells by a process similar to viral infection [4]. Finally, cationic lipid micro-bubbles called liposomes are used to deliver foreign DNA to a cell [6]. Because of opposite electrical charges, the cationic lipid encircles and packages the anionic foreign DNA. When these lipid-DNA complexes are added to cells, the lipid fuses with the membrane of the cell and delivers the foreign DNA [7]. Compared to other methods, the liposomal method generally produces a high transfection rate with very little cell mortality [7].

The major mechanical forms of transfection are electroporation, particle bombardment, and acoustically mediated transfection. Electroporation utilizes electricity to open small pores in the membrane of a cell allowing for the diffusion of DNA into the cell [8]. The particle bombardment method uses high speed projectiles with DNA to mechanically introduce the coated DNA into the cells [9, 10]. Acoustically induced transfection theoretically utilizes high energy ultrasound to disrupt the membrane of cells and allow for the uptake of DNA through diffusion [5]. This acoustic method is a relatively recent development and has been applied in mammalian cells [11, 5] and in plants [12, 13].

### BRIEF SUMMARY OF THE INVENTION

We disclose a method of transfecting mammalian or plant cells with macromolecules both in vivo and in vitro. In the in vitro method, an ultrasound signal is transmitted through the walls of normal cell medium containers, including T₂₅ flasks and six-well plates. In the in vivo method, an ultrasound signal is transmitted through a catheter-tipped transducer.

In a preferred form of the method, one first administers continuous wave ultrasound or pulse wave ultrasound to at least one cell and monitors the ultrasound using the reflected echos of the ultrasound. The cell is bathed in a cocktail containing macromolecules. A region of isonification is formed and macromolecules enter the cell at the region of the isonification. One then observes the incorporation of the macromolecules into the cell.

In a preferred form of the invention, the macromolecules are associated with bubble micronuclei, most preferably albumen microbubbles.

It is an object of the present invention to provide a transfection system for mammalian or plant cells.

It is another object of the present invention to provide a transfection system wherein albumin microbubbles enhance macromolecular transfection into plant or animal cells by ultrasound.

Other objects, advantages and features of the present invention will become apparent after examination of the specification, claims and drawings.

### BRIEF DESCRIPTION OF THE SEVERAL VIEWS OF THE DRAWINGS

FIG. 1 is a schematic diagram of the preferred embodiment of a system used to transfect cells in vitro.

FIG. 2 is a perspective view with parts cut away of the apparatus used in the system of FIG. 1 for applying ultrasonic energy to the cell cultures to be transfected.

FIG. 3 is a diagram plotting the percent of living cells transfected versus ALBUNEX concentration.

FIG. 4 is a graph plotting percent of living cells transfected versus peak negative pressure in atmospheres.

FIG. 5 is a bar chart of percent of living cells transfected versus number of exposures.

FIG. 6 is a graph plotting percent of living cells transfected versus concentration of DNA in micrograms.

FIG. 7 is a graph plotting cell mortality percentage versus ALBUNEX concentration.

FIG. 8 is a graph plotting percent of live cells transfected versus liposome concentration.

FIG. 9 is an exploded schematic diagram of a catheter for ultrasound-mediated intravascular transfection.

### DETAILED DESCRIPTION OF THE INVENTION

### 1. In General

The present invention is a method for delivery of substances to a mammalian or plant cell. Preferably, the method delivers substances to a patient. In another embodiment of the present invention, the method delivers substances to plant tissues or a whole plant. In a preferred version of the present invention, the substances are macromolecules, such as DNA, RNA, or proteins, or are therapeutic molecules.

The method comprises the steps of administering continuous wave ultrasound or pulse-wave ultrasound to a cell bathed in a cocktail containing macromolecules and monitoring the ultrasound using the reflected echos of the ultrasound. Molecules enter the cell in the isonification region and one observes incorporation of the macromolecules into the cell.

Preferably the macromolecules are part of a cocktail solution that contains bubble micronuclei. In a form of the present invention the bubble micronuclei are albumin microbubbles, preferably ALBUNEX.

In one form of the present invention, the macromolecules, most preferably DNA, are attached to the surface of the microbubbles. In another embodiment of the present invention, the DNA is within the bubbles on the interior surface. It is thought that this placement of macromolecules might decrease immune response to the treatment in patients. In another embodiment of the present invention, one might attach other therapeutic macromolecules to the bubbles, interior or exterior.

Acoustically induced transfection is based on cavitation. Cavitation refers to the formation of microbubbles of gas in a high-intensity acoustic field. Because of its relatively high frequency, ultrasound must be transmitted through a liquid medium so that it does not dissipate. Dissolved gas in this liquid medium tends to come out of solution during the low pressure stage of the acoustic wave. During the high pressure portion of the compression wave, the gas attempts to dissolve back into the solution, but because of differences in the surface area of the bubble, the bubble gains more gas during the low pressure period than it loses during the high pressure period.

With each cycle of the ultrasound wave, the bubble gains gas until it reaches equilibrium and the gases entering it are equal to the gases escaping or, alternatively, it reaches resonant diameter. If it resonant diameter, the bubble is torn apart and the energy of the acoustical field is concentrated up to 11 orders of magnitude [14]. This high concentration of power theoretically ruptures the membrane of nearby cells and allows for the passive uptake of plasmid DNA [5].Others have used ultrasound to transfect mammalian cells with very low efficiency [11].

The concentration of the microbubble nuclei in the cocktail is typically between 6×10⁶ bubbles/ml and 300×10⁶ bubbles/ml. The concentration of bubble micronuclei is typically at least 10% by volume.

The frequency of the ultrasonic waves is preferably in the range of about 0.1 to about 3.0 MHz. In one embodiment of the invention, the frequency is between 0.5-2 MHz.

Preferably, the intensity of the ultrasonic waves is in the range of 0.1-10 Watts/cm². Most preferably, the intensity is in the range of 0.1-5 or 5-10 Watts/cm².

If one wishes to transfect plants or plant cells, the intensity of the ultrasonic waves is preferably higher than 10 Watts/cm², most preferably the range would be 5-20 Watts/cm².

### B. In Vitro Transfection of Mammalian Cells

### 1. Transfection of Mammalian Cells in the Absence of Microbubbles

The majority of the work described below in this section is published as Kim, et al., Human Gene Therapy 7:1339-1346 (1996), which is hereby incorporated by reference.

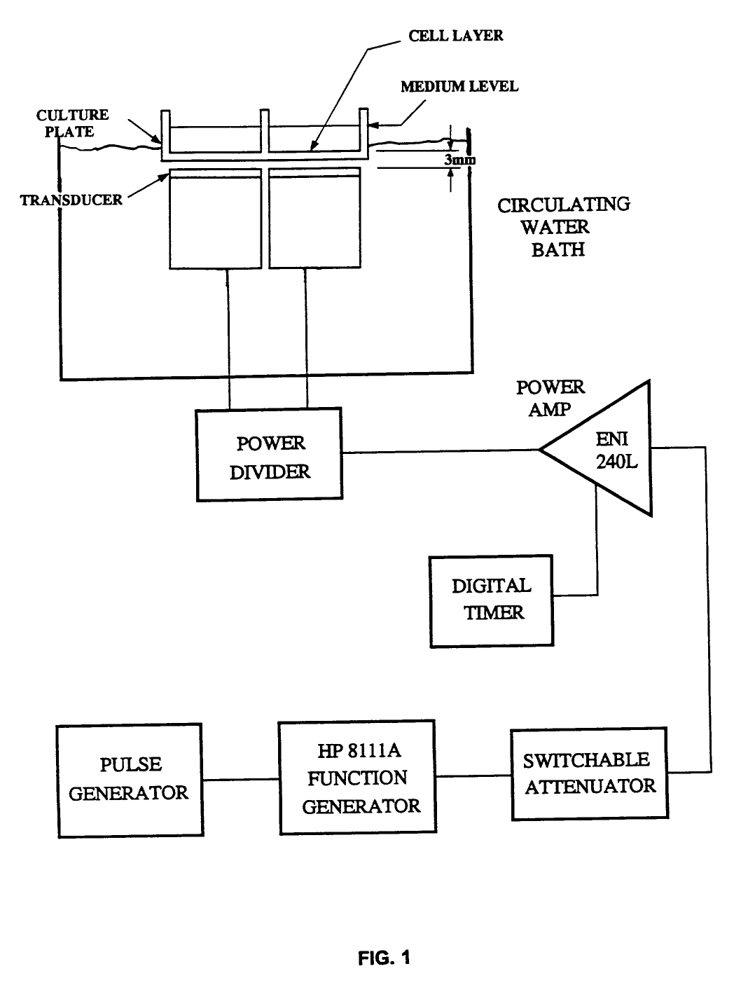

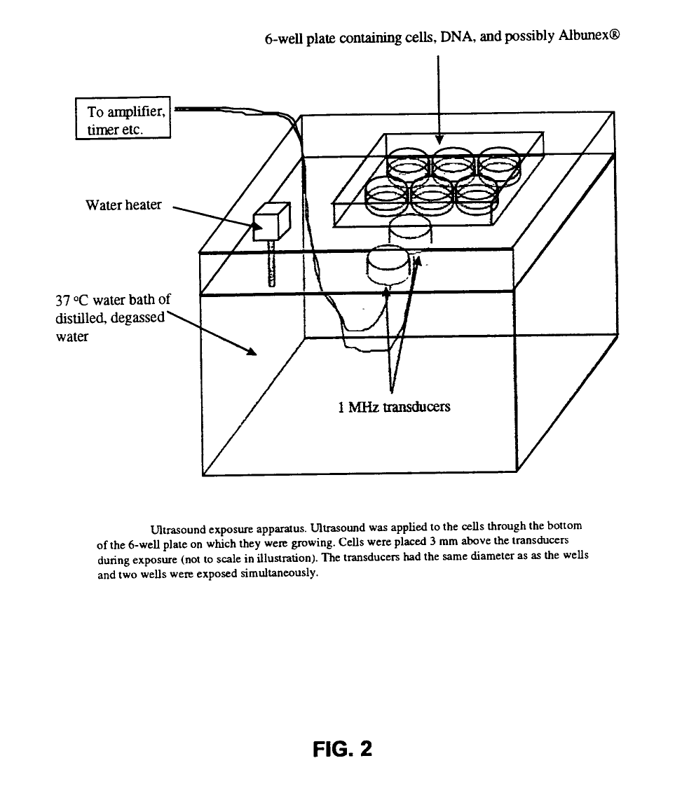

FIGS. 1 and 2 depict a preferred apparatus for application of ultrasonic waves. Referring to FIGS. 1 and 2, ultrasound of 0.1 to 3 MHz, preferably 1 MHz carrier frequency, is delivered through the bottom of a solid support, preferably a six-well plate. The ultrasound signal is in the form of continuous wave (CW) or tone pulse wave (PW). Two 35 mm diameter air-backed ultrasound transducers are fixed in a frame so that the bottoms of two adjacent wells of a six-well culture plate are aligned parallel with the transducers. The frame is placed in a water bath filled with distilled, degassed water to 8 mm above the top of the transducers. Six-well culture plates are supported on the frame 3 mm above the top of the transducers, so the ultrasound exposes the cells through the intervening distilled water and the bottom of the culture plates.

The ultrasound exposure is measured by scanning a calibrated 0.5 mm diameter hydrophone (NTR Model NP-1000, Seattle, Wash.) 4 mm in front of the transducer face in a water tank. Thus, the reported intensities are spatial average temporal peak (SATP) in the freely propagating near field of the transducer. The actual exposure conditions are standing wave conditions, because the sound reflects from the free surface of the water. Since the water ripples strongly, these standing wave conditions vary through time, producing a constant average dose throughout the media. Thus, the pressures within the media may reach twice the free field values resulting in intensities as high as four times the reported values.

We use a special shorthand notation for the ultrasound exposure values (free field) in the form XX CW for continuous wave signals (CW) or YY PWZZ for pulse wave signals (PW). In this notation an XX of 50 means 0.50 Watts/cm² intensity (SATP), YY has the same meaning for pulse wave signals, and ZZ represents the repetition frequency in Hertz (e.g. 75 PW25 for an intensity of 0.75 Watts/cm² at a repetition frequency of 25 Hz). The duty cycle (defined as the percentage of the cycle when the signal was present) for pulse waves is 20%.

The following is a method we have developed of isolating and transfecting mammalian cells. The method is easily adaptable to other cell types. In designing a method for a particular cell type, one would examine various media, exposure intensities, and length of exposure.

First, cells are harvested and cultured. For example, primary fibroblasts are harvested from hind limb muscles of seven to ten day old neonatal Long Evans rats. Fibroblasts are cultured with Dulbecco's modified Eagle medium (DMEM) containing 10% fetal bovine serum (FBS), supplemented with 10% sodium pyruvate, 10% L-glutamine, and 10% penicillin-streptomycin in a humidified incubator at 37° C. with 5% CO₂.

One then needs to prepare the substance to be transfected, preferably DNA, for transfection. In one preferred embodiment, plasmids, such as pSV-β-Galactosidase control vector (6821 bp, Promega Corp., Madison, Wis.) and pMClneo poly A (3854 bp, Stratagene Corp., La Jolla, Calif.), are used for transient and stable transfection, respectively. To amplify the plasmid, host strain bacteria are transformed using CaCl₂ and cultured in the presence of a selection agent (Ampicillin, 40 μg/ml). Amplified plasmids are purified from the bacterial cultures using a plasmid prep kit (Qiagen Inc., Chatsworth, Calif.).

Preferred molecules of the present invention include macromolecules such as DNA, RNA and protein. However, we envision other molecules, such as vitamins and other therapeutic moieties, as suitable for the present invention. Additionally, one may wish to deliver therapeutic substances, such as calcium, by the method of the present invention.

The method is then conducted according to the following procedures. Cells, such as the primary fibroblasts, are trypsinized and passaged into six-well plates at a density of 1.2×10⁵ cells/well in 2 ml of DMEM containing 10% FBS and supplements (pyruvate, glutamate, and antibiotics). Plasmid DNA is added to the media and cells are exposed to ultrasound approximately 18 hours after plating. At that time cultures are estimated to be 60% confluent.

A suitable cocktail solution of the present invention comprises microbubbles (as described below), free radical scavengers, DNAse, RNAse and protease inhibitors, and phospholipids.

In preliminary studies we determined the range of cell densities suitable for transfection. Different populations of fibroblasts are exposed to identical ultrasound conditions (1 MHz carrier frequency, 75 PW50 for three minutes) and plasmid concentrations (20 μg/ml). The number of transfected (green-stained) cells increased as the number of fibroblasts increased. The increase in the number of transfected cells was not proportional to the increase in cell number, and the efficiency (number of transfected cells/initial number of plated cells) is greatest at a concentration of 1-3×10⁵, preferably 1.2×10⁵ cells/well.

Forty-five minutes before ultrasound exposure cells are washed with phosphate buffered saline (PBS) and the media is replaced with two ml of DMEM containing plasmid but no serum or other additives. Cells are then exposed to ultrasound in a water bath pre-warmed to 37° C. Four to five minutes after ultrasound exposure, 0.25 ml of DMEM and 0.25 ml serum with 5× supplements (media with 5× serum) is added to each well. Cells are kept at 37° C. during the entire procedure. About 20 hours after ultrasound exposure, the media is replaced with fresh media containing 10% FBS and supplements.

In Kim, et al. cell survival rates are determined eight hours after ultrasound exposure by counting the number of surviving cells with a hemocytometer. Fifty-four hours after transfection cells are washed two times with PBS (pH 7.1) and fixed with glutaraldehyde solution (0.1 M sodium phosphate, pH 7.0, 1 mM MgCl₂, 0.25% glutaraldehyde) for 15 minutes at room temperature. After fixation, cells are washed three times with PBS (pH 7.1) and incubated with 1.5 ml/well of a X-gal solution (Research Products International Corp., Mount Prospect, IL) (0.2% X-gal, 1 mM MgCl₂, 150 mM NaCl, 3.3 mM K₄Fe(CN)₆0.3H₂O, 3.3 mM K₃Fe(CN)₆, 60 mM Na₂HPO₄, 40 mM NaH₂PO₄) for 3 to 3.5 hours at 37° C. After incubation, the X-gal solution was removed and cells were covered with 70% glycerol. The number of transfected cells (green stained cells expressing β-galactosidase) in each well are counted under an inverted phase contrast microscope.

Two days after transfection, cells are trypsinized and passaged into T₇₅ flasks at a density of 1×10⁴ cells/flask. Two flasks are prepared from each well. Antibiotic selection was started one day after passage by adding geneticin (SIGMA Chemical Co., St. Louis, Mo.) to the media at a final concentration of 300 μg/ml. Colonies were counted after ten days of selection. Each colony contained more than 200 transfected cells.

Initial ultrasound conditions for cell transfection were chosen on the basis of two assumptions: that ultrasound stimulated transfection on the basis of cavitation, and second, that excess permeabilization of the cell membrane to allow plasmid DNA entry results in cell death. An ultrasound carrier frequency of 1 MHz, in the form of tone bursts repeated at low frequency and 20% duty cycle or as continuous wave (CW), was chosen because these parameters have been found to enhance sonochemical effects, indicative of cavitation, in buffered solutions. The ultrasound signal intensity is chosen by examining the effect of the 1 MHz signal on cell viability. Signal intensities in the range of 0.25 to 1.0 W/cm² consistently killed 10 to 90% of exposed cells after a two minute exposure.

Further selection of the ultrasound signal is made by examining the effect of these ultrasound conditions on plasmid integrity. DNA plasmids are exposed to CW of the 1 MHz carrier at intensities of 0.25, 0.5, 0.75, and 1.0 W/cm². DNA integrity is then evaluated by gel electrophoresis and ethidium biomide staining. Degradation of the plasmid DNA is seen after 1.0 W/cm² exposure, but not after exposure to lower intensity ultrasound. Because the 1.0 W/cm² signal degraded plasmid DNA, only 0.25, 0.5, 0.75 W/cm² signals are used.

To determine the range of plasmid concentrations suitable for transfection, fibroblasts are exposed to identical ultrasound conditions at five different concentrations of pSV-β-Galactosidase (5, 10, 20, 40 and 80 μg/ml). Four transfections are performed at each plasmid concentration. The number of transfected cells/well increased as the plasmid concentration increased. Transfection at a plasmid concentration above 20 μg/ml gave the greatest efficiency.

The effect of repetition frequency on transient transfection was evaluated for 0.25, 0.5, and 0.75 W/cm² ultrasound signals. With the 0.75 and 0.5 W/cm² signals approximately 100 transfected calls are seen in both the pulsed and CW modes. No significant transfection is seen with the 0.25 W/cm² intensity signal.

Fibroblasts were exposed to identical ultrasound conditions and plasmid concentrations for times ranging from thirty seconds to seven minutes. More transfected, green-stained cells are counted with shorter exposure time (less than one minute) at 75 CW than with any exposure time at 75 PW50. Shorter exposure time (less than two minutes) at 50 CW results in more green-stained cells than at any exposure with 75 PW25.

The demonstration that transfection efficiency increased at shorter exposure times and lower intensity signals suggests that ultrasound signals cause significant cell death. The greatest numbers of transfected cells are seen after 20 or 30 seconds of exposure to 75 CW, or after 20 to 60 seconds of exposure to 50 CW. As expected, more cells are killed with increased exposure time. With either ultrasound dose the greatest number of transfected cells are seen after exposure times that result in death of approximately 50% of the cells.

The number of geneticin-resistant colonies per 1×10⁴ surviving cells ranged from 12.5 to 40.0, indicating that the highest stable transfection rate is 0.4% of surviving cells. The most efficient stable transfection occurred after 75 CW exposure for 30 seconds (average efficiency: 0.34%) followed by 75 CW with exposure time of 20 seconds, 50 CW with exposure time of 60 seconds, and 50 CW with exposure time of 40 seconds.

The selection of ultrasound conditions is based on the assumption that the critical function of the ultrasound signal is to cause cavitation, i.e., the rapid oscillation of microbubbles. The energy released during microbubble oscillation results in the plasmid DNA entering the cell, possibly by breaching the membrane and allowing molecules in the surrounding media to enter by diffusion.

### 2. Transfection of Mammalian and Plant Cells in the Presence of ALBUNEX

We envision that the transfection of mammalian and plant cells is preferably accomplished in the presence of bubble micronuclei, most preferably albumen microbubbles, such as ALBUNEX. Most of the work described below and in the Examples is taken from Greenleaf, et al., Ultrasound in Med. & Biol. 24[4]:587-595, 1998, incorporated by reference as fully set forth herein.

ALBUNEX (Mallinckrodt Medical, Inc., St. Louis, Mo., U.S.A.) is a contrast agent that is used for acoustical imaging which consists of human albumen that has been sonicated to produce micro-bubbles of gas encapsulated by albumen. ALBUNEX has been shown to increase mechanical cell damage in the presence of ultrasound in erythrocytes [15] by nucleating violent cavitational occurrences.

The Examples below describe preparation and use of ALBUNEX bubbles in the present invention.

To test the viability or efficiency of prospective methods of transfection, reporter genes are typically used. These genes have no therapeutic value but instead can be easily assayed. Reporter genes produce proteins that can be measured very accurately and/or very conveniently. Usually, these genes also produce proteins that are not normally found in the cells so that background levels of the protein are not taken as a false signal of transfection. As described above, recent work on acoustical transfection in mammalian cells was done by Kim, et al. [5] using β-galactosidase as a reporter gene and primary fibroblasts as target cells. Although β-galactosidase is a very useful marker, it requires extra steps of color development to visualize.

Green Fluorescent Protein (GFP) is a protein found in jellyfish which fluoresces under ultraviolet light [16]. The number of fluorescent, transfected cells can be assayed in a flow cytometer [17]. Flow cytometry involves the automated counting of a large number of cells. Flow cytometers can quickly count thousands of cells and record their apparent size and intensity of luminescence. Flow cytometry also produces a large amount of quantitative and objective data. The work presented below in the Examples provides general improvements of acoustically induced transfection and further extends the observations of Kim, et al. with the use of a green fluorescent protein-based reporter system, which is simple, faster, and more sensitive than the β-galactosidase-based assay.

We envision that one would attach DNA or other macromolecules to the surface of microbubbles, such as ALBUNEX, via liposomes, ionic interactions, or protein interactions.

Preferably the method of the present invention results in at least 15% of living cells transfected. More preferably, the present invention results in at least 30% of living cells transfected. In a most preferable form of the invention, the method results in approximately 60% of living cells transfected.

### 3. In Vivo Transfection of Mammalian and Plant Cells

We have developed, and described above and below, a method for transfection of plated or suspended cells. This method delivers transfection to the cells through the walls of the plate flask. Application of ultrasound from outside the cell container makes this transfection procedure more time efficient, simple, and sterile, and eliminates the requirement for special containers for the cultured cells. More importantly, transfection with an external signal suggests the possibility of noninvasive in vivo DNA transfer.

To apply the method in vivo the number of cells killed by the ultrasound exposure must be kept to a minimum. To minimize the required ultrasound pressures and thus minimize killing in vivo, we will use cavitation nuclei such as albumin microbubbles (as described above and below). These are microbubbles of air covered with a cross-linked albumin shell. They are from 1 to 8 microns in diameter. They are known to greatly decrease the threshold for cavitation which can be as high as 22 bars in pure water. Microspheres can decrease the cavitation threshold to less than 2 bars.

Another aspect of the cavitation which will be controlled in the in vivo application is the production of free radicals. Cell killing is affected by the production of free radicals in addition to the pressure in the in vivo environment. The presence of many free radical scavengers in serum and presumably the synovial space will inhibit the destructive effect of free radicals in vivo. Therefore, we will preferably add a free radical scavenger to the mixture which is injected in vivo. The components of the mixture are the plasmid, microbubbles, free radical scavenger, and DNAse inhibitor. The DNAse inhibitor is used to disable any DNAse which will break up the DNA. Of course, if one wishes to use a substance other than DNA, one may wish to use other inhibitors in the cocktail mixture, such as RNAse or protease inhibitor.

This mixture will be injected into the target area and insonated with a standing wave field. The standing wave field is produced with a large piston transducer and an ultrasound reflector such as a brass plate on the other side of the subject. The field is made to vary so that nodes and nulls of the standing wave field will not remain in one place. The entire selected volume is exposed to nodes of pressure.

### Catheter Tipped Transducer

A particularly advantageous ultrasonic and interventional catheter is described in U.S. Pat. No. 5,325,860, hereby incorporated by reference.

Cells within the body are transfected using a catheter or endoscope system to introduce both macromolecules and microbubbles for enhancing the cavitation required for ultrasonic transfection. The catheter is tipped with an ultrasound transducer that produces the appropriate signal and power level for initiating the cavitation of the nucleating agent. The cavitation thus initiated produces breaches in the walls of nearby cells and the transfection process begins. The catheter could be placed within a vessel, a body cavity, or actually inserted through a needle directly into the organ of interest. The linear or plasmid cDNA is attached to, or mixed with, the microbubble nuclei which are made with a shell of albumin. The shell stabilizes the micro-bubble and can also provide a ligand attaching surface for the specific macromolecule to be transfected into the cells.

The transducer on the tip of the catheter could be a single element transducer with focus, frequency and size all fixed for a single application. The transducer could also consist of an array of elements whereby the ultrasound energy could be focused and directed at specific regions such as lesions, neoplasms, thrombi, etc.

### External Transducer

In addition to using a catheter or endoscope tipped with an ultrasound transducer, the ultrasound source could be placed on the surface of the body whereby a focussed beam of ultrasound is directed at the region of tissue to be transfected. The microbubble/macromolecule cocktail would then be injected by needle or catheter “up stream” of the region of interest, infusing that region with the appropriate cocktail. The cocktail could be injected either within a vessel, directly within the tissue, or within a body cavity. Simultaneously, the ultrasound beam is turned on making micro-breaches in the cells so that the macromolecules traverse the cell membrane and thus transfect the cells.

### Power Level Control

The appropriate energy level required for microbubble induced transfection cavitation is much lower than that required for cavitation without microbubbles. This level of energy can be estimated with a pulse echo device associated with the transfection transducer. The pulse echo device would determine the attenuation within the tissue between the transfection transducer and the target region. The power levels and the beam shape would then be altered to produce the optimum signal for transduction at the selected region of tissue. The pulse echo device would consist of a transmitter and receiver connected to the transfection transducer. A small dose of microbubbles would be injected into the region and a sequence of pulses with increasing power is transmitted from the transduction transducer until the bubbles are destroyed by the ultrasound pulse, as detected by the disappearance of their echo signal in the receiver. This power level would be then used as a reference level to provide the level required to transfect the desired macromolecules upon their injection into the region. We have found that the threshold level of ultrasound required to break the microbubbles is below that required to transfect the cells by about 10 dB. Therefore, once the bubble breaking level is known we would turn up the power by about 10 dB and the transfecting level would be obtained in the desired region.

Microbubbles used for contrast enhancement in current clinical radiology and echocardiography are commonly seen to be destroyed by typical scanners without adverse consequences to the patient.

### Focal Control

The preliminary microbubble destruction can also be used to correct for phase or absorption aberrations of the beam. After the appropriate power level for destroying the microbubbles has been found, the focal characteristics of beam can be evaluated in the target region by noting the apparent beam positions and focal regions of microbubbles destruction. The exact region of microbubble destruction would be noted and “rehearsed” using this low level of power prior the use of the power levels required for transfection. This would provide a characterization of the beam properties in the region to be exposed for transfection and allow precise, focal transfection, unlike most other transfection methods.

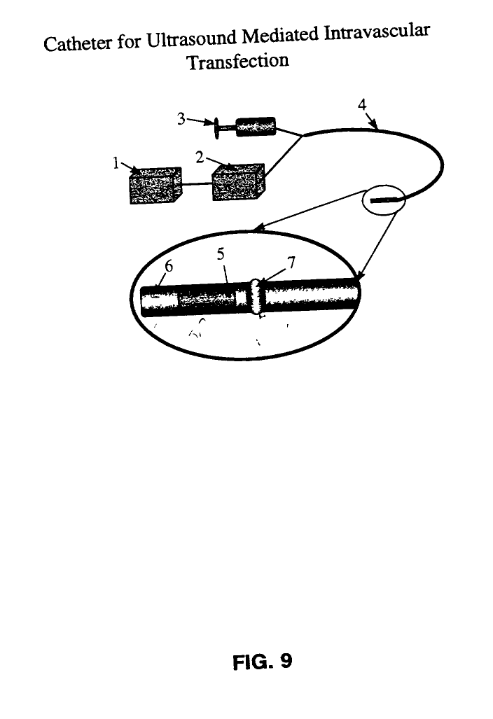

Referring to FIG. 9 there is shown in block diagram form the main components of a preferred embodiment of the site-directed transfection system which consists of a controller of the power and frequency of the ultrasound signal 1, an oscillator and associated amplifiers 2 for producing the ultrasound energy through wires connected to the ultrasound transducer 5 located at the tip of the catheter 4, and a system 3 for injecting the preferred cocktail of drugs into the catheter 4. The cocktail exits from the lumen of the catheter at an orifice 6. In addition, FIG. 9 shows a balloon 7, which can be inflated to stop the flow of blood past the catheter during its use.

The ultrasound pulses are produced after injection of the cocktail of media, plasmid/or other substances, and microbubbles. The pulses are preferably organized so as to produce about 4 bars of average peak negative pressure at the region in the tissue where the bubble nuclei are to be cavitated to cause breaches in the nearby cells for transfection. The concentration of DNA is preferably about 20-40 micrograms per ml. The concentration of microbubble nuclei is preferably about 60 million per ml.

The ultrasound signal can be either pulsed or continuous wave while the injections of cocktail can be sequential or continuous. The balloon can be on either side of the ultrasound transducer to control blood traversing either direction in the vessel.

### EXAMPLES

In these experiments, different protocols for acoustically induced transfection were tested. Many different experiments with ALBUNEX as cavitation nuclei were performed to increase the efficiency of acoustically induced transfection. Experiments with ALBUNEX included testing the effect of concentration of ALBUNEX on transfection, testing the intensity of ultrasound needed with ALBUNEX, testing multiple exposures to short bursts of ultrasound and ALBUNEX, and testing the concentration of DNA needed for ALBUNEX enhanced transfection. The following describes the general methods that were used on all cells unless otherwise specified.

Plasmid Preparation. A relatively large amount of plasmid DNA was required to test various methods of transfection. The plasmid DNA was prepared with a Qiagen 2500 μg kit according to the company's protocol (Qiagen, Inc., Chatsworth, Calif., U.S.A.). Briefly, E. coli bacteria were made to express quantities of the targeted plasmid (5.0 kbp GFP construct GreenLantern-1 from Life Technologies, Gaithersberg, Md., U.S.A.). The E. coli transformants were grown to high densities, lysed, and the lysate was passed through the Qiagen column. A special resin in the column isolated the plasmid DNA from the genomic DNA so that it could be collected separately. Finally, agarose gel electrophoresis was performed on restriction endonuclease fragments to verify the identity and purity of the plasmid DNA.

Cell Preparation. Immortalized human chondrocytes (cell line CD4 C20-A4) were either thawed (when starting from a frozen culture) or trypsinized and plated according to established protocol. Cells were plated at a concentration of 1.2×10⁶ per 6-well plate (35 mm diameter wells) (Becton Dickinson and Company, Franklin Lakes, N.J., U.S.A) and allowed to grow for about 48 hours in Dulbecco's modified Eagle medium (DMEM), containing 10% fetal bovine serum (FBS), 10% sodium pyruvate, 10% L-glutamine, and 1% penicillin-streptomycin in a 37° C. humidified incubator (5% CO₂, 95% air) until the cells were 50-70% confluent. Cells were rinsed three times with Hank's balanced salt solution, and 1 mL of DMEM (without additives) was replaced. 40 mg of plasmid DNA was then added to the DMEM.

Ultrasound Exposure. Cells were exposed to ultrasound in a 37° C. water bath. Two adjacent wells were exposed simultaneously to 1.0 MHz ultrasound at 4.0 or 3.2 atmospheres average peak pressure (unless otherwise specified) using two different 35 mm, air-backed transducers (FIG. 2). After exposure, cells were replaced into an incubator for 45 minutes and then a solution containing twice the normal concentration of FBS (20% FBS and 80% DMEM) was added. Cells were allowed to recover for 24 hours (unless otherwise noted) and were micro-photographed before counting took place to roughly determine cell mortality and so that visual comparisons could be made. Cells were then trypsinized and analyzed with a flow cytometer.

Counting of Cells. Cytometric counting was conducted on the live cells in a flow cytometer (FACScan or FACS Vantage from Becton Dickinson, San Jose, Calif., U.S.A). The GFP used in this experiment had maximum excitation of 490 nm and emits light in the 520 nm range (according to the manufacturer). Through the flow cytometer, the cells were exposed to 488 nm light and were detected at 530 nm plus or minus 15 nm. The background level of fluorescence was determined by assaying cells that had not been experimentally manipulated. This background was subtracted from the experimental cell counts and the number of transfected cells was determined. In most cases, 10,000 cells were counted. After flow cytometric analysis, the cells were preserved with 2% formalin.

Acoustically Induced Transfection in the Presence of ALBUNEX. ALBUNEX was tested for its potential to enhance acoustically induced transfection. ALBUNEX was added to DMEM immediately before exposure at concentrations of 1%, 10%, and 50% and 5%, 10%, 20%. The exposure conditions of Kim, et al. were used in these experiments. Briefly, these conditions were exposure to 1.0 MHz ultrasound at 4.0 or 3.2 atmospheres for 20 seconds. Previous experiments have shown negligible uptake of naked DNA with no ultrasound.

Acoustically Induced Transfection with ALBUNEX at Various Intensities and Repetitions of Exposure. ALBUNEX was tested for enhancement of acoustically induced transfection at different intensities and with repetitions of exposure. Six pressures were tested: 2.8, 2.2, 2.0, 1.6, 1.4 and 1.1 atmospheres. Repetitive 1.0 second exposures to 4.0 atmosphere ultrasound and ALBUNEX were also investigated. After each exposure to ultrasound, fresh ALBUNEX (10% of volume) was added to the medium before re-exposure (DNA was not re-added prior to re-exposure). Exposures of 2.0 seconds at 2.0 or 1.6 atmospheres and 20 seconds at 2.0 or 1.6 atmospheres were also carried out so that certain extrapolations could be calculated.

Effect of DNA Concentration on Acoustically Induced Transfection. Finally, DNA concentrations were tested for their effect on transfection. Cells were treated with DNA concentrations of 100, 25, 10, 5, and 2 micrograms of DNA per well. Also, optimal liposomal concentrations and protocols were confirmed and photographs were taken for visual comparison to acoustically induced transfection.

### Results

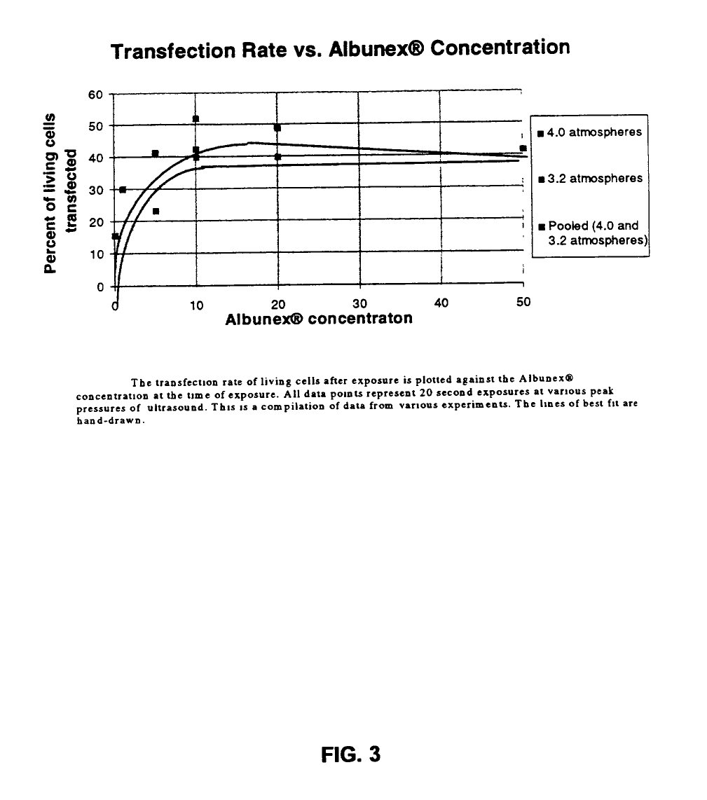

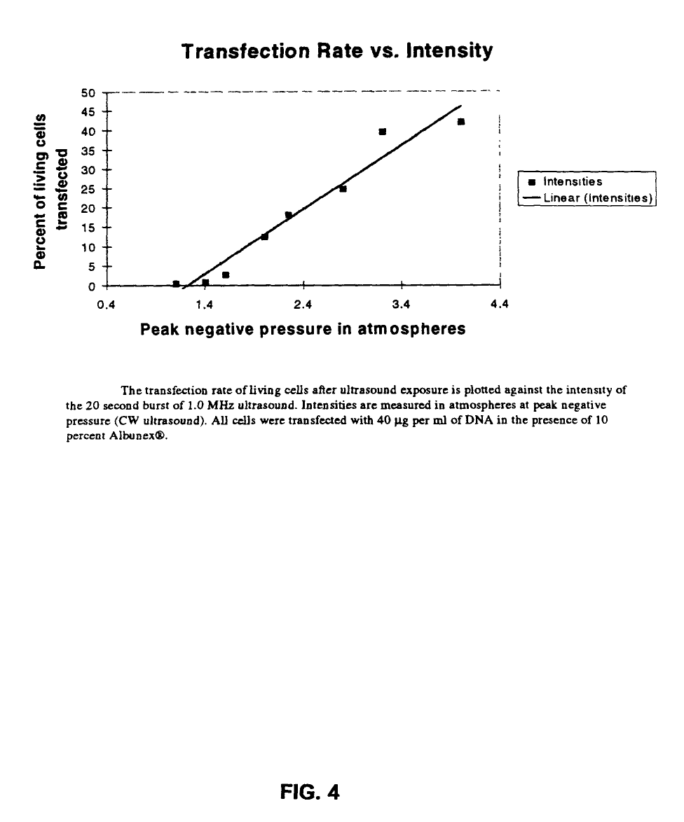

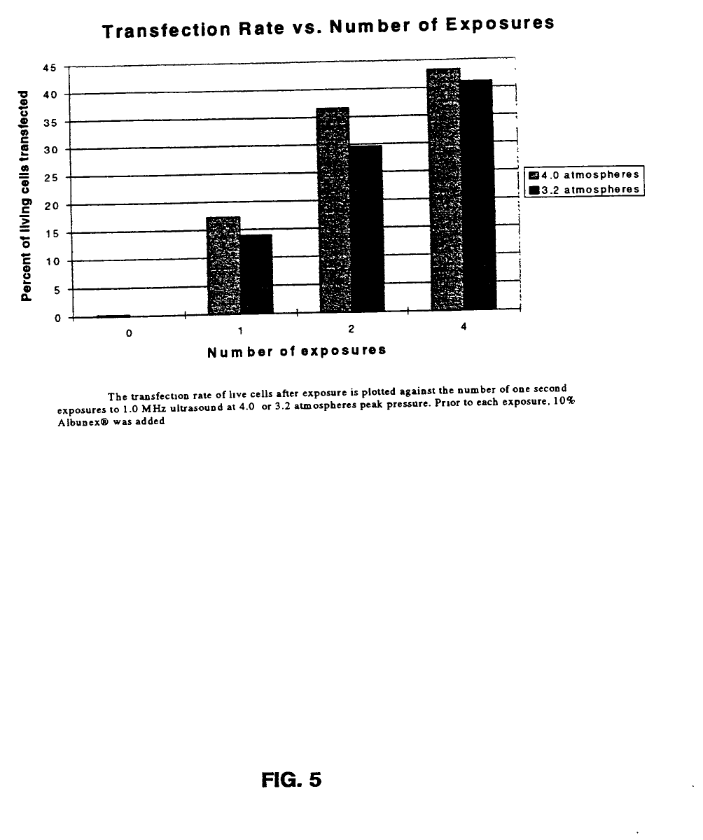

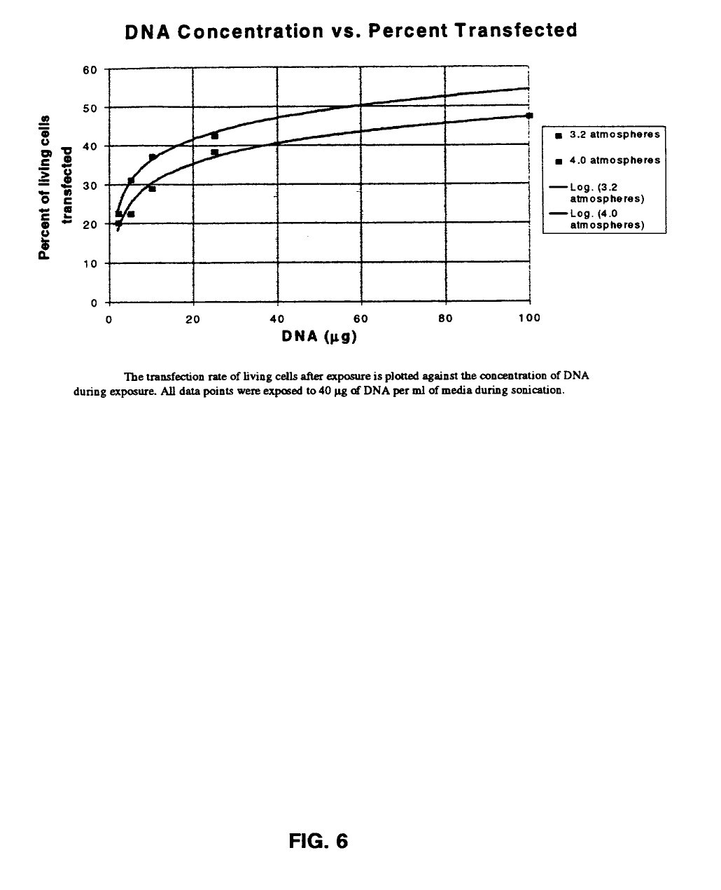

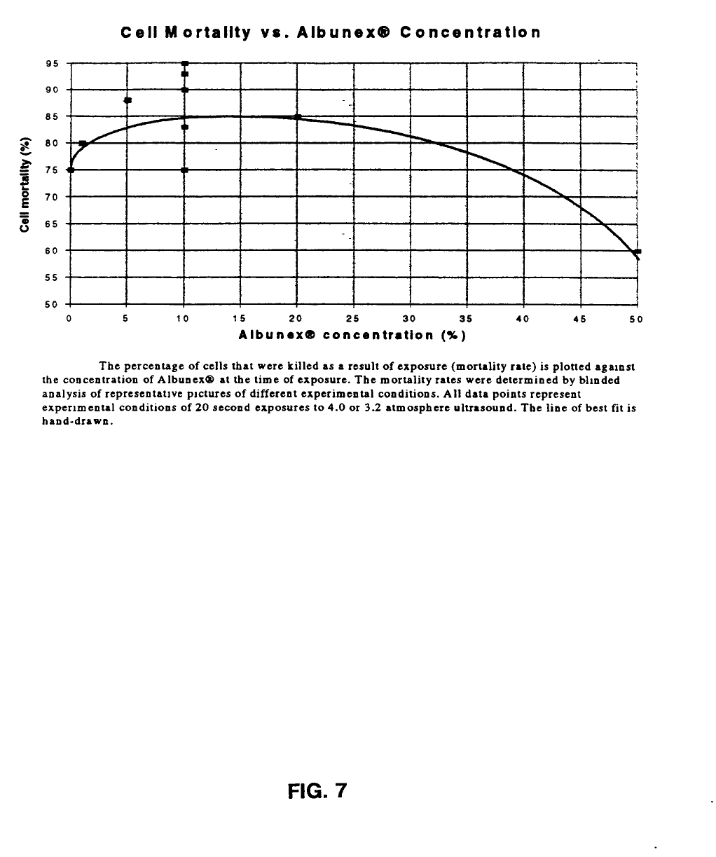

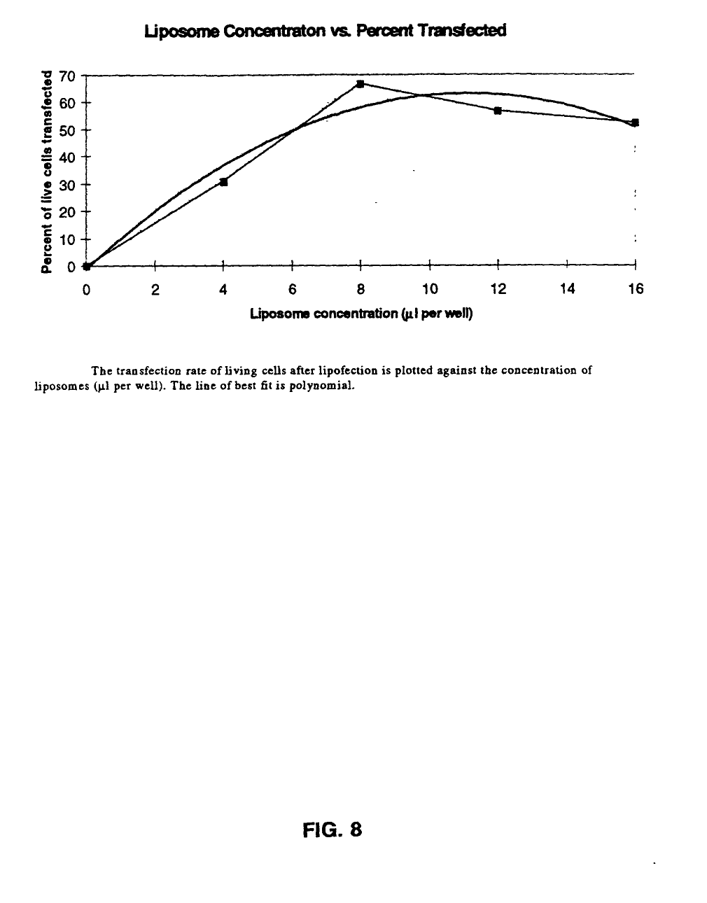

The relationship between the concentration of ALBUNEX and the transfection efficiency of live cells is shown in FIG. 3. The maximum efficiency is seen to be near 10% ALBUNEX by volume. Results presented in FIG. 4 show a fairly linear relationship between transfection efficiency and intensity of ultrasound. Results presented in FIG. 5 show that dosages of 10% ALBUNEX transfect cells with minimal ultrasound exposure. This effect is fairly cumulative for one and two exposures to 1 second of ultrasound, but this cumulative property is not present when extrapolated to four exposures. Experiment four shows that this ALBUNEX-enhanced procedure is DNA dose dependent (FIG. 6). Data from subjective mortality evaluation are shown in FIG. 7 and are compiled from various experiments. The liposome experiment shows that the optimal concentration of liposomes is near 10-12 micrograms per ml (FIG. 8).

### Discussion

ALBUNEX had a marked effect on the transfection efficiency of acoustically induced transfection. ALBUNEX effectively doubled or tripled the transfection efficiency of the original method as described in Kim, et al. and performed in this experiment with GFP and immortalized chondrocytes. Visually and microscopically, it can be seen that the ALBUNEX is destroyed immediately after the application of high intensity ultrasound (all intensities studied in these experiments). Repetitive exposure experiments show that transfection can also occur solely through destruction of ALBUNEX. In other words, pulses of high intensity ultrasound through ALBUNEX will transfect cells alone without the twenty second exposure as specified in Kim, et al. These data show that through the addition of cavitation nuclei, transfection is increased. This finding substantiates the theory that cavitation is an important factor for transfection. These experiments also suggest that DNA concentration and transfection efficiency are related, but are not linearly proportional. As DNA concentration is increased, the transfection efficiency is also increased, and eventually plateaued correlating to a logarithmic relationship (FIG. 6). The introduction of ALBUNEX drastically changed the transfection curve from previous DNA concentration experiments (not shown). It significantly increased the efficiency of low amounts of DNA and shifted the line of best fit to the left. However, these previous results were obtained using β-galactosidase in primary chondrocytes, and because of differences discussed later, the results may be skewed. Kim, et al. also used β-gal to measure transfection in primary chondrocytes and found a transfection efficiency of about 2.4%. Using the same protocol, except for the use of GFP, immortalized chondrocytes, and flow cytometric counting, a transfection rate of around 15% was obtained in experiments presented here. This contributes to evidence that these vectors give wildly different strengths of signal, that the flow cytometer is better at counting positive cells, or that immortalized chondrocytes are more susceptible to this form of transfection. Other experiments show that with decreased intensity of exposure, transfection also decreases. Even at these lower intensities, the ALBUNEX is ruptured (visually and microscopically verified). Because rupturing of the albumen micro-bubbles does not necessarily equate with transfection, the violence of the rupturing of the ALBUNEX may have a significant impact on transfection efficiency. Experimental evidence suggests that at high intensities the ALBUNEX may burst violently, concentrating enough energy to rupture the cell membrane. At lower intensities, however, the ALBUNEX many rupture more sedately and have little effect on the cell. This is an area of further study.

Comparison to Other Methods and Applications. Acoustically induced transfection is fairly comparable to other high performance forms of transfection such as lipofection. There are many possible applications of this method of transfection. Because this transfection technique can be performed through the walls of plastic lab ware, it reduces the possibility of contamination compared to many other methods. Another interesting possibility in the application of this form of transfection is with regard to plants. Because of the intense mechanical means of initiating transfection, this method should work on cells with tough extra-cellular matrixes or cell walls. Other purely mechanical techniques such as particle bombardment work well in plants [9]. Theoretically, the intensity of the ultrasound can be increased so that destruction of the ALBUNEX occurs with enough violence to open the plant cell wall and allow uptake of foreign DNA.

Final Comments. Finally, the possibility of stable transfection through acoustically induced transfection warrants further research. Kim, et al. found a 0.34% stable transfection rate in fibroblasts. The effect of ALBUNEX on stable transfection was not investigated in this study and may be an avenue for further research.

Unfortunately, considerable cell death does occur as a result of this procedure (60-90% mortality). The inherently violent nature of this procedure makes it prone to high rates of cell mortality. This may be a major obstacle if the method were to be successfully applied. Further, suitable optimization experiments would be required to determine the precise limit to cell durability. However, particle bombardment, another purely mechanical transfection technique, has been successfully applied in vivo [4]. Another drawback to this procedure is the relatively large amounts of plasmid DNA that are needed to obtain competitive transfection rates. However, as commercial plasmid preparation kits become widespread and the price of plasmid DNA drops dramatically, this obstacle can be easily overcome.

Conclusion. Significant enhancement of acoustically induced transfection was observed through the use of cavitation nuclei in the form of the contrast agent ALBUNEX. ALBUNEX is effective at lower intensities and shorter exposure times than were formerly required. ALBUNEX also seems to have increased the efficiency of low doses of DNA over previous results. Repetitive, short exposures were seen to have a nearly cumulative effect on transfection. Also, it appears that the ultrasonic pressure with which the ALBUNEX is destroyed has an effect on transfection. Although significant cell death occurs, this form of transfection was shown to transfect upwards of 50% of the living cells after exposure, which is comparable to other transfection techniques such as lipofection. Overall, the addition of micro-bubble cavitation nuclei significantly enhances acoustically induced transfection over what has been previously reported.

### REFERENCES

[1] E. G. Hanania, J. Kavanagh, G. Hortolbagyi, and R. Giles. Recent advances in the application of gene therapy to human disease. The American Journal of Medicine, 99:537-552.

[2] S. Akli, J-E. Guidotti, E. Vigne, M. Perricaudet, K. Sandhoff, A. Kahn, and L. Poenaru. Restoration of hexosaminidase a activity in human tay-sachs fibroblasts via adenoviral vector-mediated gene transfer. Gene Therapy, 3:769-774, 1996.

[3] Hrouda and A. G. Dalgleish. Gene therapy for prostate cancer. Gene Therapy, 3:845-852, 1996.

[4] N-S. Yang. Gene transfer into mammalian somatic cells. Critical Reviews in Biotechnology, 12(4):335-356, 1992.

[5] H. J. Kim, J. F. Greenleaf, R. R. Kinnick, J. T.

Bronk, and M. E. Bolander. Ultrasound mediated transfection of mammalian cells. Human Gene Therapy, 7:1339-1346, Jul. 10, 1996.

[6] Y. Liu, D. Liggitt, W. Zhong, T. Guanhuan, K.

Gaensler, and R. Debs. Cationic liposome-mediated intravenous gene delivery. The Journal of Biological Chemistry, 270(42):24864-24870.

[7] H. Gershon, R. Ghirlando, S. B. Guttman, and A. Minsky. Mode of formation and structural features of DNA-cationic liposome complexes used for transfection. Biochemistry, 32:7143-7151, 1993.

[8] D. C. Chang and T. S. Reese. Changes in membrane structure induced by electroporation as revealed by rapid-freezing electron microscopy. Biophysics Journal, 58:1-12, 1990.

[9] H. Daniell. Foreign gene expression in chloroplasts of higher plants mediated by tungsten particle bombardment. Methods in Enzymology, 217:536-557, 1993.

[10] N-S. Yang, J. Birkkholder, B. Roberts, B. Martinell, and D. McCabe. In vivo and in vitro gene transfer to mammalian somatic cells by particle bombardment. Proceedings of the National Academy of Science, 87:9568-9572, 1990.

[11] M. Fechheimer, J. F. Boyan, S. Parker, J. E. Sisken, G. L. Partel, and S. G. Zimmer. Transfection of mammalian cells with plasmid DNA by scrape loading and sonication loading. Proceedings of the National Academy of Sciences, USA, 84:8463-8467, 1987.

[12] M. Joersbo and J. Brunstedt. Direct gene transfer to plant protoplasts by mild sonication. Plant Cell Reports, 9:207-210, 1990.

[13] M. Joersbo and J. Brunstedt. Sonication: A new method for gene transfer to plants. Physiol. Plant., 85:230-234, 1992.

[14] L. A. Crum, R. A. Roy, M. A. Dinno, C. C. Church, R. E. Apfel, C. K. Holland, and S. I. Madanshetty. Acoustic cavitation produced by microsecond pulses of ultrasound: a discussion of some selected results. Journal of the Acoustical Society of America, 91:1113-1119, 1992.

[15] M. W. Miller and A. Brayman. Biophysics team investigates the interaction of cavitation bubbles and suspended cells in vitro. Technical report, Rochester Center for Biomedical Ultrasound, 1993.

[16] S. R. Kain, M. Adams, A. Kondepudi, T.-T. Yang, W. W. Ward, and P. Kitts. Green fluorescent protein as a reporter of gene expression and protein localization. BioTechniques, 19:650-655, 1995.

[17] D. W. Galbraith, G. M. Lambert, R. J. Grebenok, and J. Sheen. Flow cytometric analysis of transgene expression in higher plants: Green-fluorescent protein. Methods in Cell Biology, 50:3-14, 1995.

We claim:

1. A method for delivery of macromolecules to a cell comprising:
(a) administering continuous wave ultrasound or pulsed wave ultrasound to at least one cell wherein the cell is bathed in a cocktail solution comprising a substance to be transfected;
(b) monitoring the ultrasound using the reflected echoes of the ultrasound, wherein a region of isonification is formed; wherein the substance enter the cell in the region of the isonification; and
(c) observing incorporation of the substance into the cell.

2. The method of claim 1 wherein the cocktail comprises bubble micronuclei.

3. The method of claim 2 wherein the bubble micronuclei are albumen microbubbles.

4. The method of claim 3 wherein the albumen microbubbles are ALBUNEX.

5. The method of claim 2, wherein the concentration of microbubble nuclei in the cocktail is between 6×10⁶ bubbles/ml and 300×10⁶ bubbles/ml.

6. The method of claim 2 wherein the concentration of bubble micronuclei is between 1% and 10% by volume.

7. The method of claim 1 wherein a controller is used for injecting the cocktail.

8. The method of claim 1, wherein the ultrasound is directed at the specified tissue region within the patient using an external transducer.

9. The method of claim 8 wherein the transducer is also used in an imaging mode.

10. The method of claim 1, wherein the cocktail is injected into the region of ultrasound exposure by the lumen of a catheter.

11. The method of claim 10 wherein the injection is by a hypodermic needle.

12. The method of claim 10, wherein the catheter additionally comprises a balloon capable of stopping blood or urine flow by being inflated prior to injection of the cocktail and exposure to ultrasound.

13. The method of claim 2 wherein the size of the microbubbles ranges from about 5.0 to 10.0 microns.

14. The method of claim 2 wherein the size of the microbubbles ranges from about 0.5 to 5 microns.

15. The method of claim 2 wherein DNA is attached to the surface of the micronuclei.

16. The method of claim 5 wherein the micronuclei are targeted to specific cell receptors using antigens.

17. The method of claim 1 wherein the frequency of the ultrasonic waves is in the range of about 0.01 to about 1.0 MHz.

18. The method of claim 1 wherein the frequency of the ultrasonic waves is in the range of about 1.0 to about 3.0 MHz.

19. The method of claim 1 wherein the intensity of the ultrasonic waves is in the range of 0.1 to 5 Watts/cm².

20. The method of claim 1 wherein the range is 5 to 10 Watts/cm².

21. The method of claim 8 wherein the transducer element consists of more than one element arranged so as to make a directed beam.

22. The method of claim 8 wherein the material for transduction of electric to ultrasound energy is piezoelectric.

23. The method of claim 8 wherein the material for transduction of electric to ultrasound energy is selected from the group consisting of magnetostrictive and electrostrictive materials.

24. The method of claim 8 wherein the material for transduction of electric to ultrasound energy is pneumatic.

25. The method of claim 1 wherein the injection is automatic.

26. The method of claim 2 wherein the microbubbles are coated with phospholipids.

27. The method of claim 2 wherein the microbubbles are coated with human albumen.

28. The method of claim 1 wherein the ultrasound signal comprises two or more combined frequencies.

29. The method of claim 1 wherein the ultrasound signal comprises short pulses or tone bursts between 0.1 and 2.0 seconds in duration.

30. The method of claim 1 wherein the cells are mammalian.

31. The method of claim 30 wherein the cells are within a patient.

32. The method of claim 1 wherein the cells are plant cells.

33. The method of claim 32 wherein the cells are part of a plant tissue.

34. The method of claim 1 where bubbles are coated with cell specific receptor binding complexes.

35. The method of claim 31 wherein the cells are bathed in a liquid medium and wherein the temperature of the medium is between 20° C. and 60° C.

36. The method of claim 1 wherein the substance is a macromolecule.

37. The method of claim 36 wherein the substance is DNA.

38. The method of claim 3 wherein the micronuclei are coated with the substance to be transfected.
